(19) United States
(12) Patent Application Publication
(10) Pub. No.: US 2025/0277779 A1
(43) Pub. Date: Sep. 4, 2025

(54) SILICA IN ROCK SAMPLES

(71) Applicant: Chevron U.S.A. Inc., San Ramon, CA (US)

(72) Inventors: Eliza MATHIA, Birmington (GB); Paul Montgomery, Bricklehampton (GB); Kenneth Thomas RATCLIFFE, Welshpool (GB)

(73) Assignee: Chevron U.S.A. Inc., San Ramon, CA (US)

(21) Appl. No.: 19/052,693

(22) Filed: Feb. 13, 2025

Related U.S. Application Data
(62) Division of application No. 18/003,420, filed on Dec. 27, 2022.

(51) Int. Cl. G01N 33/24; G01N 21/35; G01N 21/3563; G01N 23/223

(52) U.S. Cl. CPC G01N 33/24; G01N 21/3563; G01N 23/223; G01N 2021/3595; G01N 2201/127

(57) ABSTRACT

A method comprises determining an amount of excess silica and/or an amount of terrigenous silica in a rock sample taking into account a measurement indicative of an amount of silica in the rock sample and a measurement indicative of an amount of zirconium in the rock sample.

### CROSS-REFERENCE TO RELATED APPLICATIONS

The present application is a division of U.S. patent application Ser. No. 18/003,420 filed Dec. 27, 2022, entitled “Silica In Rock Samples” [pending], which is a national phase application, filed under 35 U.S.C. 371, of International Patent Application Number PCT/US2020/040533 filed Jul. 1, 2020.

### FIELD

The present disclosure concerns methods of determining amounts of excess silica, terrigenous silica, biogenic-excess silica and/or authigenic-excess silica in rock samples, as well as associated computer programs, computer-readable media and data carrier signals.

### BACKGROUND

Rock samples obtained from sedimentary basins typically contain silica. The silica in a sedimentary rock sample may be present in different forms. For example, sedimentary rock samples may contain silica in the form of terrigenous or detrital silica, which is silica incorporated in detrital sedimentary grains (e.g detrital quartz or feldspar grains) in the rock. Detrital sedimentary grains derive from material liberated by the weathering and erosion of pre-existing rock in the basin hinterland, transported into the basin by rivers and wind. Sedimentary rock samples may also contain silica which is not incorporated in detrital grains; this is known as excess silica. Excess silica in a rock sample can be further categorised as authigenic-excess silica or biogenic-excess silica. Authigenic-excess silica is silica that has been liberated from detrital sedimentary material by chemical weathering and precipitated within the rock as silica (typically, quartz) cement. Biogenic-excess silica is silica that has been liberated from silica-rich marine organisms (e.g. plankton), such as diatoms, radiolaria, silicoflagellates and siliceous sponges, by chemical weathering and precipitated within the rock as silica (typically, quartz) cement.

Hydrocarbon explorers have found that the quality of hydrocarbon reservoirs in a region correlates with the type of silica deposits in that region. For example, relatively higher proportions of excess silica, in comparison to terrigenous silica, are associated with increased hydrocarbon production. Rock parameters such as the porosity and/or mechanical properties (e.g. Young's modulus) of rocks also depend on the relative amounts of biogenic-excess silica and authigenic-excess silica in the rock. For example, biogenic-excess silica is typically more brittle than authigenic-excess silica and therefore can be easier to fracture, for example when forming hydrocarbon wells by hydraulic fracturing.

The composition of rock samples extracted from hydrocarbon wells can be determined precisely in the laboratory, for example using methods such as quantitative X-ray diffraction (QXRD), combustion analysis (e.g. using a LECO combustion instrument), pyrolysis analysis (e.g. using a Rock-Eval pyrolysis instrument) or isotope analysis (e.g. silicon isotope analysis). However, such analytical methods are typically time-consuming and can require the use of high-quality core samples or thin sections.

Improved methods for analysing the silica content of rock samples would therefore be of benefit.

### SUMMARY

In a first aspect, a method comprises determining an amount of excess silica and/or an amount of terrigenous silica in a rock sample (e.g. a sedimentary rock sample, i.e. a sample of sedimentary rock) taking into account a measurement indicative of (e.g. measurement of) an amount of silica (i.e. silicon dioxide (SiO₂)) in the rock sample and a measurement indicative of (e.g. measurement of) an amount of zirconium (i.e. Zr) in the rock sample.

It will be appreciated that terrigenous silica in a rock sample obtained from a sedimentary basin is silica in the rock sample which derives from weathering (i.e. erosion) of the hinterland (i.e. surroundings) of the sedimentary basin at the time of rock formation. Terrigenous silica in the rock sample is therefore silica in the rock sample originally derived from terrestrial, as opposed to marine or lacustrine, environments.

It will further be appreciated that the total silica content of a rock sample obtained from a sedimentary basin can be divided between terrigenous silica and excess silica (i.e. the total silica content consists of terrigenous silica and excess silica).

In particular, there are two primary types of excess silica in rock samples obtained from sedimentary basins: authigenic-excess silica and biogenic-excess silica. Authigenic-excess silica is silica that has been liberated from detrital sedimentary material by chemical weathering and precipitated within the rock as silica (typically, quartz) cement. Biogenic-excess silica is silica that has been liberated from silica-rich marine organisms (e.g. plankton), such as diatoms, radiolaria, silicoflagellates and siliceous sponges, by chemical weathering and precipitated within the rock as silica (typically, quartz) cement. It may be that the amount of excess silica in the rock sample is a parameter indicative of a volume (e.g. total volume) of excess silica in the rock sample. The parameter indicative of the volume (e.g. total volume) of excess silica in the rock sample may be the volume (e.g. total volume) of excess silica in the rock sample. Alternatively, the parameter indicative of the volume of excess silica in the rock sample may be a volume fraction (e.g. a volume percentage) of excess silica in the rock sample (e.g. the fraction (e.g. percentage) of the total volume of the rock sample consisting of excess silica).

Alternatively, it may be that the amount of excess silica in the rock sample is a parameter indicative of a mass (e.g. total mass) of excess silica in the rock sample. The parameter indicative of the mass (e.g. total mass) of excess silica in the rock sample may be the mass (e.g. total mass) of excess silica in the rock sample. Alternatively, the parameter indicative of the mass of excess silica in the rock sample may be a mass fraction (e.g. a mass percentage) of excess silica in the rock sample (e.g. the fraction (e.g. percentage) of the total mass of the rock sample consisting of excess silica).

It may be that the amount of terrigenous silica in the rock sample is a parameter indicative of a volume (e.g. total volume) of terrigenous silica in the rock sample. The parameter indicative of the volume (e.g. total volume) of terrigenous silica in the rock sample may be the volume (e.g. total volume) of terrigenous silica in the rock sample. Alternatively, the parameter indicative of the volume of terrigenous silica in the rock sample may be a volume fraction (e.g. a volume percentage) of terrigenous silica in the rock sample (e.g. the fraction (e.g. percentage) of the total volume of the rock sample consisting of terrigenous silica).

Alternatively, it may be that the amount of terrigenous silica in the rock sample is a parameter indicative of a mass (e.g. total mass) of terrigenous silica in the rock sample. The parameter indicative of the mass (e.g. total mass) of terrigenous silica in the rock sample may be the mass (e.g. total mass) of terrigenous silica in the rock sample.

Alternatively, the parameter indicative of the mass of terrigenous silica in the rock sample may be a mass fraction (e.g. a mass percentage) of terrigenous silica in the rock sample (e.g. the fraction (e.g. percentage) of the total mass of the rock sample consisting of terrigenous silica).

It may be that the amount of silica in the rock sample is a parameter indicative of a volume (e.g. total volume) of silica in the rock sample. The parameter indicative of the volume (e.g. total volume) of silica in the rock sample may be the volume (e.g. total volume) of silica in the rock sample. Alternatively, the parameter indicative of the volume of silica in the rock sample may be a volume fraction (e.g. a volume percentage) of silica in the rock sample (e.g. the fraction (e.g. percentage) of the total volume of the rock sample consisting of silica).

Alternatively, it may be that the amount of silica in the rock sample is a parameter indicative of a mass (e.g. total mass) of silica in the rock sample. The parameter indicative of the mass (e.g. total mass) of silica in the rock sample may be the mass (e.g. total mass) of silica in the rock sample. Alternatively, the parameter indicative of the mass of silica in the rock sample may be a mass fraction (e.g. a mass percentage) of silica in the rock sample (e.g. the fraction (e.g. percentage) of the total mass of the rock sample consisting of silica).

It will be understood that the amount of silica in the rock sample is the total amount of silica in the rock sample, i.e. including all types of silica in the rock sample, i.e. including both excess silica (i.e. including both biogenic-excess silica and authigenic-excess silica) and terrigenous silica.

It may be that the amount of zirconium in the rock sample is a parameter indicative of a volume (e.g. total volume) of zirconium in the rock sample. The parameter indicative of the volume (e.g. total volume) of zirconium in the rock sample may be the volume (e.g. total volume) of zirconium in the rock sample. Alternatively, the parameter indicative of the volume of zirconium in the rock sample may be a volume fraction (e.g. a volume percentage) of zirconium in the rock sample (e.g. the fraction (e.g. percentage) of the total volume of the rock sample consisting of zirconium).

Alternatively, it may be that the amount of zirconium in the rock sample is a parameter indicative of a mass (e.g. total mass) of zirconium in the rock sample. The parameter indicative of the mass (e.g. total mass) of zirconium in the rock sample may be the mass (e.g. total mass) of zirconium in the rock sample. Alternatively, the parameter indicative of the mass of zirconium in the rock sample may be a mass fraction (e.g. a mass percentage) of zirconium in the rock sample (e.g. the fraction (e.g. percentage) of the total mass of the rock sample consisting of zirconium).

The method may comprise obtaining the measurement indicative of (e.g. measurement of) the amount of silica in the rock sample and/or the measurement indicative of (e.g. measurement of) the amount of zirconium in the rock sample from the rock sample. That is to say, the method may comprise measuring the amount of silica in the rock sample and/or measuring the amount of zirconium in the rock sample. Measuring the amount of silica in the rock sample may comprise measuring an amount of silicon in the rock sample and determining (e.g. estimating or calculating) the amount of silica in the rock sample based on the measured amount of silicon in the rock sample (for example, assuming that all silicon found in the rock sample is present in the form of silica).

It may be that the measurement indicative of (e.g. measurement of) the amount of silica in the rock sample and/or the measurement indicative of (e.g. measurement of) the amount of zirconium in the rock sample are spectroscopic measurements such as X-ray spectroscopic measurements. That is to say, the method may comprise measuring the amount of silica and/or the amount of zirconium in the rock sample by a spectroscopic method such as an X-ray spectroscopic method.

It will be appreciated that a spectroscopic measurement method is a method for compositional (e.g. elemental or chemical) analysis of a material based on the analysis of a spectrum (e.g. an electromagnetic spectrum) generated by the material, particularly when exposed to a source of energy such as electromagnetic radiation or a particle flux (e.g. an electron beam). It will further be appreciated that an X-ray spectroscopic measurement method is a method for elemental or chemical analysis of a material based on the analysis of an X-ray spectrum generated by the material, particularly when exposed to a source of energy such as electromagnetic radiation (e.g. X-rays) or an electron beam. Suitable X-ray spectroscopic methods include wavelength-dispersive X-ray spectroscopic methods (i.e. WDX) and energy-dispersive X-ray spectroscopic methods (i.e. EDX), for example using X-ray fluorescence (XRF), in which X-rays are used to excite electronic transitions to generate an X-ray emission spectrum, or an electron microprobe or scanning electron microscope (SEM), in which an electron beam is used to excite electronic transitions to generate an X-ray emission spectrum.

Accordingly, it may be that the measurement indicative of (e.g. measurement of) the amount of silica in the rock sample and/or the measurement indicative of (e.g. measurement of) the amount of zirconium in the rock sample are X-ray fluorescence (XRF) measurements. That is to say, the method may comprise measuring the amount of silica in the rock sample by XRF and/or measuring the amount of zirconium in the rock sample by XRF. Measuring the amount of silica in the rock sample by XRF may comprise measuring the amount of silicon in the rock sample by XRF and determining (e.g. estimating or calculating) the amount of silica in the rock sample based on the measured amount of silicon in the rock sample (for example, assuming that all silicon found in the rock sample is present in the form of silica). X-ray fluorescence may be particularly suitable for use in the field due to the availability of portable, hand-held XRF devices.

It may be that determining the amount of excess silica in the rock sample and/or the amount of terrigenous silica in the rock sample comprises taking into account a relationship between measurements indicative of (e.g. measurements of) amounts of terrigenous silica and measurements indicative of (e.g. measurements of) amounts of zirconium for rock samples. For example, the rock sample may derive from (i.e. have been obtained from) a particular region (e.g. a particular sedimentary basin or a particular area within the sedimentary basin) and determining the amount of excess silica in the rock sample and/or the amount of terrigenous silica in the rock sample may comprise taking into account a relationship between measurements indicative of (e.g.

measurements of) amounts of terrigenous silica and measurements indicative of (e.g. measurements of) amounts of zirconium for rock samples in the said region (e.g. in the said sedimentary basin or area within the sedimentary basin).

Zirconium in sedimentary rocks typically derives from erosion of the hinterland surrounding the sedimentary basin. Moreover, the zirconium content of sedimentary rocks was not typically enriched by prehistoric biological activity, nor was zirconium typically dissolved in prehistoric bodies of waters such as lakes or oceans in sedimentary basins. Accordingly, the abundance of zirconium in a sedimentary rock is typically dependent on the proportion of the rock which is of terrigenous origin. Zirconium content may therefore be used a proxy for quantifying the proportion of the rock which is of terrigenous origin.

In particular, the inventors have found that the amount of terrigenous silica in a rock sample and the amount of zirconium in the said rock sample are typically in direct proportion to one another. That is to say, there is typically a linear relationship between the amount of terrigenous silica in a rock sample and the amount of zirconium in the said rock sample.

Accordingly, it may be that the relationship between measurements indicative of (e.g. measurements of) amounts of terrigenous silica and measurements indicative of (e.g. measurements of) amounts of zirconium for rock samples (e.g. in a region, for example in a sedimentary basin or area within the sedimentary basin) is a ratio between amounts of terrigenous silica and measurements indicative of (e.g. measurements of) amounts of zirconium for rock samples (e.g. in the region, for example in the sedimentary basin or area within the sedimentary basin).

The method may comprise: determining an amount of terrigenous silica in the rock sample taking into account the measurement indicative of (e.g. measurement of) the amount of zirconium in the rock sample and the relationship (e.g. the linear relationship, for example the ratio) between measurements indicative of (e.g. measurements of) amounts of terrigenous silica and measurements indicative of (e.g. measurements of) amounts of zirconium for rock samples (e.g. in the region, for example in the sedimentary basin or area within the sedimentary basin); and determining the amount of excess silica in the rock sample based on the determined amount of terrigenous silica in the rock sample and the measurement indicative of (e.g. measurement of) the amount of silica in the rock sample. The method may comprise determining the amount of excess silica in the rock sample as the difference between the measurement indicative of (e.g. measurement of) the amount of silica in the rock sample and the determined amount of terrigenous silica in the rock sample.

The rock sample may be a core sample. The skilled person will appreciate that a core sample is a cylindrical section of rock having standardised dimensions. For example, a core sample may be a cylindrical section of rock having a diameter of about 1 inch. Plugs may be extracted from core samples for detailed analysis.

Alternatively, the rock sample may be a cuttings sample. The skilled person will appreciate that a cuttings sample is a sample of drill cuttings obtained when a well is drilled. Drill cuttings typically comprise (e.g. consist of) relatively small, broken pieces of rock produced by drilling action and brought to the surface in drilling mud. Cuttings samples are commonly examined as part of mud logging (i.e. well logging) processes.

It may be that one or more steps of the method are carried out by a computer. For example, it may be that the method comprises the computer determining the amount of excess silica and/or the amount of terrigenous silica in the rock sample taking into account the measurement indicative of (e.g. measurement of) the amount of silica in the rock sample and the measurement indicative of (e.g. measurement of) the amount of zirconium in the rock sample. It may be that the method comprises the computer determining the amount of excess silica in the rock sample and/or the amount of terrigenous silica in the rock sample taking into account the relationship between measurements indicative of (e.g. measurements of) amounts of terrigenous silica and measurements indicative of (e.g. measurements of) amounts of zirconium for rock samples. It may be that the method comprises the computer: determining the amount of terrigenous silica in the rock sample taking into account the measurement indicative of (e.g. measurement of) the amount of zirconium in the rock sample and the relationship between measurements indicative of (e.g. measurements of) amounts of terrigenous silica and measurements indicative of (e.g. measurements of) amounts of zirconium for rock samples; and determining the amount of excess silica in the rock sample based on the determined amount of terrigenous silica in the rock sample and the measurement indicative of (e.g. measurement of) the amount of silica in the rock sample.

In a second aspect, a computer program comprises instructions which, when the program is executed by a computer, cause the computer to carry out the steps of the method according to the first aspect. For example, it may be that the instructions, when the program is executed by the computer, cause the computer to carry out any combination of the steps of the method of the first aspect identified hereinabove as being carried out by (or being suitable for being carried out by) a computer.

In a third aspect, there is provided a (e.g. non-transitory) computer-readable medium storing the computer program (e.g. the instructions) according to the second aspect. The computer program (e.g. the instructions) may be stored as computer-executable program code.

In a fourth aspect, there is provided a data carrier signal carrying (e.g. encoding) the computer program (e.g. the instructions) according to the second aspect. The computer program (e.g. the instructions) may be provided in the form of computer-executable program code.

In a fifth aspect, a method comprises determining an amount of biogenic-excess silica and/or an amount of authigenic-excess silica in a rock sample taking into account a measurement indicative of (e.g. measurement of) an amount of excess silica in the rock sample, a measurement indicative of (e.g. measurement of) an amount of carbon in the rock sample and a measurement indicative of (e.g. measurement of) an amount of nitrogen in the rock sample.

It may be that the amount of biogenic-excess silica in the rock sample is a parameter indicative of a volume (e.g. total volume) of biogenic-excess silica in the rock sample. The parameter indicative of the volume (e.g. total volume) of biogenic-excess silica in the rock sample may be the volume (e.g. total volume) of biogenic-excess silica in the rock sample. Alternatively, the parameter indicative of the volume of biogenic-excess silica in the rock sample may be a volume fraction (e.g. a volume percentage) of biogenic-excess silica in the rock sample (e.g. the fraction (e.g. percentage) of the total volume of the rock sample consisting of biogenic-excess silica).

Alternatively, it may be that the amount of biogenic-excess silica in the rock sample is a parameter indicative of a mass (e.g. total mass) of biogenic-excess silica in the rock sample. The parameter indicative of the mass (e.g. total mass) of biogenic-excess silica in the rock sample may be the mass (e.g. total mass) of biogenic-excess silica in the rock sample. Alternatively, the parameter indicative of the mass of biogenic-excess silica in the rock sample may be a mass fraction (e.g. a mass percentage) of biogenic-excess silica in the rock sample (e.g. the fraction (e.g. percentage) of the total mass of the rock sample consisting of biogenic-excess silica).

It may be that the amount of authigenic-excess silica in the rock sample is a parameter indicative of a volume (e.g. total volume) of authigenic-excess silica in the rock sample. The parameter indicative of the volume (e.g. total volume) of authigenic-excess silica in the rock sample may be the volume (e.g. total volume) of authigenic-excess silica in the rock sample. Alternatively, the parameter indicative of the volume of authigenic-excess silica in the rock sample may be a volume fraction (e.g. a volume percentage) of authigenic-excess silica in the rock sample (e.g. the fraction (e.g. percentage) of the total volume of the rock sample consisting of authigenic-excess silica).

Alternatively, it may be that the amount of authigenic-excess silica in the rock sample is a parameter indicative of a mass (e.g. total mass) of authigenic-excess silica in the rock sample. The parameter indicative of the mass (e.g. total mass) of authigenic-excess silica in the rock sample may be the mass (e.g. total mass) of authigenic-excess silica in the rock sample. Alternatively, the parameter indicative of the mass of authigenic-excess silica in the rock sample may be a mass fraction (e.g. a mass percentage) of authigenic-excess silica in the rock sample (e.g. the fraction (e.g. percentage) of the total mass of the rock sample consisting of authigenic-excess silica).

It may be that the amount of excess silica in the rock sample is a parameter indicative of a volume (e.g. total volume) of excess silica in the rock sample. The parameter indicative of the volume (e.g. total volume) of excess silica in the rock sample may be the volume (e.g. total volume) of excess silica in the rock sample. Alternatively, the parameter indicative of the volume of excess silica in the rock sample may be a volume fraction (e.g. a volume percentage) of excess silica in the rock sample (e.g. the fraction (e.g. percentage) of the total volume of the rock sample consisting of excess silica).

Alternatively, it may be that the amount of excess silica in the rock sample is a parameter indicative of a mass (e.g. total mass) of excess silica in the rock sample. The parameter indicative of the mass (e.g. total mass) of excess silica in the rock sample may be the mass (e.g. total mass) of excess silica in the rock sample. Alternatively, the parameter indicative of the mass of excess silica in the rock sample may be a mass fraction (e.g. a mass percentage) of excess silica in the rock sample (e.g. the fraction (e.g. percentage) of the total mass of the rock sample consisting of excess silica).

It may be that the amount of carbon in the rock sample is an amount of organic carbon in the rock sample. It will be appreciated that organic carbon is carbon derived from organic, i.e. biological, sources, in comparison to inorganic carbon derived from inorganic sources. Examples of organic carbon include hydrocarbons (e.g. free hydrocarbons), kerogen, bitumen and pyrobitumen. The amount of organic carbon in the rock sample may be the amount of total organic carbon (TOC) in the rock sample, wherein TOC comprises all sources of organic carbon in the rock sample.

It may be that the amount of carbon (e.g. organic carbon, for example total organic carbon) in the rock sample is a parameter indicative of a volume (e.g. total volume) of carbon (e.g. organic carbon, for example total organic carbon) in the rock sample. The parameter indicative of the volume (e.g. total volume) of carbon (e.g. organic carbon, for example total organic carbon) in the rock sample may be the volume (e.g. total volume) of carbon (e.g. organic carbon, for example total organic carbon) in the rock sample. Alternatively, the parameter indicative of the volume of carbon (e.g. organic carbon, for example total organic carbon) in the rock sample may be a volume fraction (e.g. a volume percentage) of carbon (e.g. organic carbon, for example total organic carbon) in the rock sample (e.g. the fraction (e.g. percentage) of the total volume of the rock sample consisting of carbon (e.g. organic carbon, for example total organic carbon)).

Alternatively, it may be that the amount of carbon (e.g. organic carbon, for example total organic carbon) in the rock sample is a parameter indicative of a mass (e.g. total mass) of carbon (e.g. organic carbon, for example total organic carbon) in the rock sample. The parameter indicative of the mass (e.g. total mass) of carbon (e.g. organic carbon, for example total organic carbon) in the rock sample may be the mass (e.g. total mass) of carbon (e.g. organic carbon, for example total organic carbon) in the rock sample. Alternatively, the parameter indicative of the mass of carbon (e.g. organic carbon, for example total organic carbon) in the rock sample may be a mass fraction (e.g. a mass percentage) of carbon (e.g. organic carbon, for example total organic carbon) in the rock sample (e.g. the fraction (e.g. percentage) of the total mass of the rock sample consisting of carbon (e.g. organic carbon, for example total organic carbon)).

It may be that the amount of nitrogen in the rock sample is an amount of organic nitrogen in the rock sample. It will be appreciated that organic nitrogen is carbon derived from organic, i.e. biological, sources, in comparison to inorganic nitrogen derived from inorganic sources.

It may be that the amount of nitrogen (e.g. organic nitrogen) in the rock sample is a parameter indicative of a volume (e.g. total volume) of nitrogen (e.g. organic nitrogen) in the rock sample. The parameter indicative of the volume (e.g. total volume) of nitrogen (e.g. organic nitrogen) in the rock sample may be the volume (e.g. total volume) of nitrogen (e.g. organic nitrogen) in the rock sample. Alternatively, the parameter indicative of the volume of nitrogen (e.g. organic nitrogen) in the rock sample may be a volume fraction (e.g. a volume percentage) of nitrogen (e.g. organic nitrogen) in the rock sample (e.g. the fraction (e.g. percentage) of the total volume of the rock sample consisting of nitrogen (e.g. organic nitrogen)).

Alternatively, it may be that the amount of nitrogen (e.g. organic nitrogen) in the rock sample is a parameter indicative of a mass (e.g. total mass) of nitrogen (e.g. organic nitrogen) in the rock sample. The parameter indicative of the mass (e.g. total mass) of nitrogen (e.g. organic nitrogen) in the rock sample may be the mass (e.g. total mass) of nitrogen (e.g. organic nitrogen) in the rock sample. Alternatively, the parameter indicative of the mass of nitrogen (e.g. organic nitrogen) in the rock sample may be a mass fraction (e.g. a mass percentage) of nitrogen (e.g. organic nitrogen) in the rock sample (e.g. the fraction (e.g. percentage) of the total mass of the rock sample consisting of nitrogen (e.g. organic nitrogen)).

The method may comprise obtaining the measurement indicative of (e.g. measurement of) the amount of excess silica and/or the measurement indicative of (e.g. measurement of) the amount of carbon (e.g. organic carbon, for example TOC) and/or the measurement indicative of (e.g. measurement of) the amount of nitrogen (e.g. organic nitrogen) from the rock sample. That is to say, the method may comprise measuring the amount of excess silica and/or the amount of carbon (e.g. organic carbon, for example TOC) and/or the amount of nitrogen (e.g. organic nitrogen) in the rock sample

It may be that the measurement indicative of (e.g. measurement of) the amount of excess silica in the rock sample is obtained by the method according to the first aspect and/or using the computer program according to the second aspect. Accordingly, the method may comprise measuring the amount of excess silica in the rock sample using the method according to the first aspect and/or using the computer program according to the second aspect.

It may be that the measurement indicative of (e.g. measurement of) the amount of carbon (e.g. organic carbon, for example TOC) in the rock sample and/or the measurement indicative of (e.g. measurement of) the amount of nitrogen (e.g. organic nitrogen) in the rock sample are spectroscopic measurements such as infra-red spectroscopic measurements. That is to say, the method may comprise measuring the amount of carbon (e.g. organic carbon, for example TOC) and/or the amount of nitrogen (e.g.

organic nitrogen) in the rock sample by a spectroscopic method such as an infra-red spectroscopic method.

It will be appreciated that a spectroscopic measurement method is a method for compositional (e.g. elemental or chemical) analysis of a material based on the analysis of a spectrum (e.g. an electromagnetic spectrum) generated by the material, particularly when exposed to a source of energy such as electromagnetic radiation (e.g. infra-red radiation) or a particle flux (e.g. an electron beam).

It may be that the measurement indicative of (e.g. measurement of) the amount of carbon (e.g. organic carbon, for example TOC) in the rock sample and/or the measurement indicative of (e.g. measurement of) the amount of nitrogen (e.g. organic nitrogen, for example TOC) in the rock sample are Fourier Transform Infra-Red (FTIR) spectroscopic measurements. That is to say, the method may comprise measuring the amount of carbon (e.g. organic carbon, for example TOC) in the rock sample by FTIR spectroscopy and/or measuring the amount of nitrogen (e.g. organic nitrogen) in the rock sample by FTIR spectroscopy.

It may be that obtaining the measurement indicative of (e.g. measurement of) the amount of carbon (e.g. organic carbon, for example TOC) in the rock sample and/or the measurement indicative of (e.g. measurement of) the amount of nitrogen (e.g. organic nitrogen) in the rock sample comprises: obtaining a spectroscopic measurement (e.g. an FTIR spectroscopic measurement) from the rock sample; and determining the amount of carbon (e.g. organic carbon, for example TOC) and/or the amount of nitrogen (e.g. organic nitrogen) in the rock sample based on the spectroscopic measurement (e.g. FTIR spectroscopic measurement) and a spectroscopic calibration model (e.g. an FTIR spectroscopic calibration model) which defines a relationship between spectroscopic measurements (e.g. FTIR spectroscopic measurements) and amounts of carbon (e.g. organic carbon, for example TOC) and/or amounts of nitrogen (e.g. organic nitrogen) for rock samples.

The spectroscopic measurement may comprise (e.g. be) a value of a spectroscopic parameter (for example, an emission or absorption signal (e.g. intensity) at a particular wavelength) or a plurality of values of a spectroscopic parameter (for example, emission or absorption signals (e.g. intensities) at a plurality of different wavelengths), e.g. a spectroscopic (emission or absorption) spectrum.

The spectroscopic calibration model may define a mathematical relationship (e.g. a functional relationship or mapping) between the spectroscopic measurements and the amounts of carbon (e.g. organic carbon, for example TOC) and/or amounts of nitrogen (e.g. organic nitrogen) for rock samples. The spectroscopic calibration model may therefore be (or be represented by) a mathematical function. The mathematical function may be expressed (or expressible) in an analytical or a numerical form. The mathematical function may be parameterised based on (i.e. in terms of) spectroscopic calibration model parametrisation data, for example as stored in a look-up table.

It may be that determining the amount of biogenic-excess silica in the rock sample and/or the amount of authigenic-excess silica in the rock sample comprises taking into account a relationship between measurements indicative of (e.g. measurements of) amounts of biogenic-excess silica and/or authigenic-excess silica and measurements indicative of (e.g. measurements of) amounts of carbon (e.g. organic carbon, for example TOC) and measurements indicative of (e.g. measurements of) amounts of nitrogen (e.g. organic nitrogen) for rock samples. For example, the rock sample may derive from (i.e. have been obtained from) a particular region (e.g. a particular sedimentary basin or a particular area within the sedimentary basin) and determining the amount of biogenic-excess silica in the rock sample and/or the amount of authigenic-excess silica in the rock sample may comprise taking into account a relationship between measurements indicative of (e.g. measurements of) amounts of biogenic-excess silica and/or authigenic-excess silica and measurements indicative of (e.g. measurements of) amounts of carbon (e.g. organic carbon, for example TOC) and measurements indicative of (e.g. measurements of) amounts of nitrogen (e.g. organic nitrogen) for rock samples in the said region (e.g. in the said sedimentary basin or area within the sedimentary basin).

In particular, the inventors have found that the amount of carbon (e.g. organic carbon, for example TOC) and the amount of nitrogen (e.g. organic nitrogen) in a rock sample is indicative of the level of primary productivity (i.e. the rate at which plants and other photosynthetic organisms produced organic compounds in the prehistoric ecosystem) at the time the rock was formed. Accordingly, the inventors have found that the abundance of biogenic-excess silica in a rock sample typically correlates with the amount of carbon (e.g. organic carbon, for example TOC) and the amount of nitrogen (e.g. organic nitrogen) in a rock sample. More particularly, the inventors have found that the amount of biogenic-excess silica in a rock sample correlates with a ratio of the amount of organic carbon (e.g. TOC) to the amount of organic nitrogen in the rock sample. In contrast, the amount of authigenic-excess silica in a rock sample is not strongly dependent on the amount of carbon (e.g. organic carbon, for example TOC) and the amount of nitrogen (e.g. organic nitrogen) in a rock sample, or more particularly, not strongly dependent the ratio of the amount of organic carbon (e.g. TOC) to the amount of organic nitrogen in the rock sample. However, because excess silica typically consists of biogenic-excess silica and authigenic-excess silica, and because the abundance of biogenic-excess silica in a rock sample depends on the abundance of carbon (e.g. organic carbon, for example TOC) and the abundance of nitrogen (e.g. organic nitrogen) in the rock sample, the proportion of the excess silica in a rock sample which consists of authigenic-excess silica does typically depend on the abundance of carbon (e.g. organic carbon, for example TOC) and the abundance of nitrogen (e.g. organic nitrogen) in the rock sample.

Accordingly, it may be that the relationship between measurements indicative of (e.g. measurements of) amounts of biogenic-excess silica and/or authigenic-excess silica and measurements indicative of (e.g. measurements of) amounts of carbon (e.g. organic carbon, for example TOC) and measurements indicative of (e.g. measurements of) amounts of nitrogen (e.g. organic nitrogen) for rock samples is a relationship between measurements indicative of (e.g. measurements of) amounts of biogenic-excess silica and/or authigenic-excess silica and measurements indicative of (e.g. measurements of) a ratio of amounts of carbon (e.g. organic carbon, for example TOC) to amounts of nitrogen (e.g. organic nitrogen) for rock samples.

The method may comprise determining a ratio of the amount of biogenic-excess silica in the rock sample to the amount of authigenic-excess silica in the rock sample taking into account the relationship between measurements indicative of (e.g. measurements of) amounts of biogenic-excess silica and/or authigenic-excess silica and measurements indicative of (e.g. measurements of) amounts of carbon (e.g. organic carbon, for example TOC) and measurements indicative of (e.g. measurements of) amounts of nitrogen (e.g. organic nitrogen) for rock samples.

The method may comprise: determining the ratio of the amount of biogenic-excess silica in the rock sample to the amount of authigenic-excess silica in the rock sample taking into account the relationship between measurements indicative of (e.g. measurements of) amounts of biogenic-excess silica and/or authigenic-excess silica and measurements indicative of (e.g. measurements of) amounts of carbon (e.g. organic carbon, for example TOC) and measurements indicative of (e.g. measurements of) amounts of nitrogen (e.g. organic nitrogen) for rock samples; and determining the amount of biogenic-excess silica and/or the amount of authigenic-excess silica in the rock sample based on (a) the determined ratio of the amount of biogenic-excess silica in the rock sample to the amount of authigenic-excess silica in the rock sample and (b) the measurement indicative of (e.g. measurement of) the amount of excess silica in the rock sample.

The method may comprise determining the amount of biogenic-excess silica and/or the amount of authigenic-excess silica in the rock sample using a graphical method.

The rock sample may be a core sample. The skilled person will appreciate that a core sample is a cylindrical section of rock having standardised dimensions. For example, a core sample may be a cylindrical section of rock having a diameter of about 1 inch. Plugs may be extracted from core samples for detailed analysis.

Alternatively, the rock sample may be a cuttings sample. The skilled person will appreciate that a cuttings sample is a sample of drill cuttings obtained when a well is drilled. Drill cuttings typically comprise (e.g. consist of) relatively small, broken pieces of rock produced by drilling action and brought to the surface in drilling mud. Cuttings samples are commonly examined as part of mud logging (i.e. well logging) processes.

It may be that one or more steps of the method are carried out by a computer. For example, it may be that the method comprises the computer determining the amount of biogenic-excess silica and/or the amount of authigenic-excess silica in the rock sample taking into account the measurement indicative of (e.g. measurement of) the amount of excess silica in the rock sample, the measurement indicative of (e.g. measurement of) the amount of carbon (e.g. organic carbon, for example TOC) in the rock sample and the measurement indicative of (e.g. measurement of) the amount of nitrogen (e.g. organic nitrogen) in the rock sample. The method may comprise the computer determining the amount of biogenic-excess silica in the rock sample and/or the amount of authigenic-excess silica in the rock sample taking into account the relationship between measurements indicative of (e.g. measurements of) amounts of biogenic-excess silica and/or authigenic-excess silica and measurements indicative of (e.g. measurements of) amounts of carbon (e.g. organic carbon, for example TOC) and measurements indicative of (e.g. measurements of) amounts of nitrogen (e.g. organic nitrogen) for rock samples. The method may comprise the computer determining the ratio of the amount of biogenic-excess silica in the rock sample to the amount of authigenic-excess silica in the rock sample taking into account the relationship between measurements indicative of (e.g. measurements of) amounts of biogenic-excess silica and/or authigenic-excess silica and measurements indicative of (e.g. measurements of) amounts of carbon (e.g. organic carbon, for example TOC) and measurements indicative of (e.g. measurements of) amounts of nitrogen (e.g. organic nitrogen) for rock samples. The method may comprise the computer determining the amount of biogenic-excess silica and/or the amount of authigenic-excess silica in the rock sample based on (a) the determined ratio of the amount of biogenic-excess silica in the rock sample to the amount of authigenic-excess silica in the rock sample and (b) the measurement indicative of (e.g. measurement of) an amount of excess silica in the rock sample. The method may comprise the computer determining the amount of biogenic-excess silica and/or the amount of authigenic-excess silica in the rock sample using the graphical method.

In a sixth aspect, there is provided a computer program comprising instructions which, when the program is executed by a computer, cause the computer to carry out the steps of the method of the fifth aspect. For example, it may be that the instructions, when the program is executed by the computer, cause the computer to carry out any combination of the steps of the method of the fifth aspect identified hereinabove as being carried out by (or being suitable for being carried out by) a computer.

In a seventh aspect, there is provided a (e.g. non-transitory) computer-readable medium storing the computer program (e.g. the instructions) according to the sixth aspect. The computer program (e.g. the instructions) may be stored as computer-executable program code.

In an eighth aspect, there is provided a data carrier signal carrying (e.g. encoding) the computer program (e.g. the instructions) according to the sixth aspect. The computer program (e.g. the instructions) may be provided in the form of computer-executable program code.

In a ninth aspect, a method comprises: fitting a spectroscopic calibration model to spectroscopic measurement data and corresponding carbon (e.g. organic carbon, for example TOC) and/or nitrogen (e.g. organic nitrogen) compositional measurement data obtained from a plurality of (i.e. different) reference rock samples, wherein the spectroscopic calibration model defines a relationship between the spectroscopic measurement data and the corresponding carbon (e.g. organic carbon, for example TOC) and/or nitrogen (e.g. organic nitrogen) compositional measurement data for the plurality of reference rock samples.

Accordingly, it may be that the method comprises: providing the spectroscopic measurement data obtained from the plurality of (i.e. different) reference rock samples; and providing the corresponding carbon (e.g. organic carbon, for example TOC) and/or nitrogen (e.g. organic nitrogen) compositional measurement data obtained from the (i.e. same) plurality of (i.e. different) reference rock samples. The method may further comprise: obtaining the spectroscopic measurement data from the plurality of (i.e. different) reference rock samples (i.e. using a spectroscopic method); and obtaining the corresponding carbon (e.g. organic carbon, for example TOC) and/or nitrogen (e.g. organic nitrogen) compositional measurement data from the (i.e. same) plurality of (i.e. different) reference rock samples.

The spectroscopic measurement data may be infra-red spectroscopic measurement data such as Fourier Transform Infra-Red (FTIR) spectroscopic measurement data. Accordingly, the spectroscopic calibration model may be an FTIR spectroscopic calibration model, wherein the FTIR spectroscopic calibration model defines a relationship between the FTIR spectroscopic measurement data and the corresponding carbon (e.g. organic carbon, for example TOC) and/or nitrogen (e.g. organic nitrogen) compositional measurement data for the plurality of reference rock samples. For example, the spectroscopic calibration model may be an FTIR spectroscopic calibration model, wherein the FTIR spectroscopic calibration model defines a relationship between the FTIR spectroscopic measurement data and the corresponding carbon (e.g. organic carbon, for example TOC) and/or nitrogen (e.g. organic nitrogen) compositional measurement data for the plurality of reference rock samples.

The carbon (e.g. organic carbon, for example TOC) and/or nitrogen (e.g. organic nitrogen) compositional measurement data may be obtained by combustion analysis (for example using a combustion analyser such as available from LECO Corporation, Saint Joseph, Michigan, USA) of the reference rock samples. Accordingly, the carbon (e.g. organic carbon, for example TOC) compositional measurement data may comprise measurements indicative of (e.g. measurements of) the amount of carbon (e.g. organic carbon, for example TOC) in each reference rock sample, for example combustion analysis measurements (e.g. LECO measurements) of the amount of carbon (e.g. organic carbon, for example TOC) in each reference rock sample. The amount of organic carbon (e.g. TOC) in each reference rock sample may be measured by combustion analysis following removal of inorganic carbon (e.g. carbonates), for example by dissolution. The nitrogen (e.g. organic nitrogen) compositional measurement data may comprise measurements indicative of (e.g. measurements of) the amount of nitrogen (e.g. organic nitrogen) in each reference rock sample, for example combustion analysis measurements (e.g. LECO measurements) of the amount of nitrogen (e.g. organic nitrogen) in each reference rock sample.

One or more steps of the method may be carried out by a computer. For example, the method may comprise the computer fitting the spectroscopic calibration model to the spectroscopic measurement data and the corresponding carbon (e.g. organic carbon, for example TOC) and/or nitrogen (e.g. organic nitrogen) compositional measurement data. The spectroscopic calibration model may be fitted to the spectroscopic measurement data and the corresponding carbon (e.g. organic carbon, for example TOC) and/or nitrogen (e.g. organic nitrogen) compositional measurement data using a multivariate statistical method such as a least squares regression method. However, the skilled person will appreciate that any other suitable fitting methods as known in the art may be used. For example, the spectroscopic calibration model may be fitted to the spectroscopic measurement data and the corresponding carbon (e.g. organic carbon, for example TOC) and/or nitrogen (e.g. organic nitrogen) compositional measurement data using a machine learning algorithm (for example, using a neural network or a genetic algorithm) as known in the art.

In a tenth aspect, a computer program comprises instructions which, when the program is executed by a computer, cause the computer to carry out one or more steps of the method according to the ninth aspect. For example, it may be that the instructions, when the program is executed by the computer, cause the computer to carry out any combination of the steps of the method of the ninth aspect identified hereinabove as being carried out by (or being suitable for being carried out by) a computer.

In an eleventh aspect, there is provided a data set comprising the spectroscopic measurement data (e.g. FTIR spectroscopic measurement data) of the ninth aspect and/or the carbon (e.g. organic carbon, for example TOC) and/or nitrogen (e.g. organic nitrogen) compositional measurement data of the ninth aspect and/or spectroscopic calibration model parameter data (e.g. FTIR spectroscopic calibration model parameter data) on which (i.e. in terms of which) the spectroscopic calibration model (e.g. FTIR spectroscopic calibration model) of the ninth aspect is parametrised.

In a twelfth aspect, there is provided a (e.g. non-transitory) computer-readable medium storing the computer program (e.g. the instructions) according to the tenth aspect and/or the data set according to the eleventh aspect. The computer program (e.g. the instructions) may be stored as computer-executable program code.

In a thirteenth aspect, there is provided a data carrier signal carrying (e.g. encoding) the computer program (e.g. the instructions) according to the tenth aspect and/or the data set according to the eleventh aspect. The computer program (e.g. the instructions) may be provided in the form of computer-executable program code.

The skilled person will appreciate that, except where mutually exclusive, a feature described in relation to any one of the above aspects may be applied mutatis mutandis to any other aspect. Furthermore, except where mutually exclusive, any feature described herein may be applied to any aspect and/or combined with any other feature described herein.

### FIGURES

Embodiments will now be described by way of example only, with reference to the Figures, in which:

FIG. 1 is a plot of total silica content versus zirconium content for a plurality of rock samples from the same sedimentary basin;

FIG. 2A shows the ratio of organic carbon to organic nitrogen (i.e., the C/N ratio) and the excess silica content for a plurality of rock samples extracted from a well in the Permian basin, as a function of core depth within the well;

FIG. 2B shows the excess silica content versus the C/N ratio for the said samples;

FIG. 3 is a plot of total organic carbon content versus total organic nitrogen content for a plurality of rock samples from the same sedimentary basin;

FIG. 4 is a plot of excess silica content versus C/N ratio for the rock samples of FIG. 3;

FIG. 5 is a plot of the data shown in FIG. 4 following translation such that authigenic silica and biogenic silica boundary lines intersect at the origin;

FIG. 6 is a plot of authigenic-excess silica content versus biogenic-excess silica content for the rock samples of FIGS. 3 to 5;

FIG. 7 is an example FTIR absorption spectrum from a sedimentary rock sample;

FIG. 8 is a plot comparing measurements of the weight fraction of total organic carbon (TOC) in the same rock samples using Rock-Eval pyrolysis and calibrated Fourier-transform infra-red spectroscopy (FTIR);

FIG. 9 shows a computer processor in communication with a computer-readable medium storing a computer program comprising computer-executable instructions; and

FIG. 10 shows plots of silica content versus zirconium content for shale rock samples extracted from the Muskwa formation in Canada and the Haynesville formation in the United States of America, indicating biogenic and terrestrial trends.

### DETAILED DESCRIPTION

### Rocks

Rocks are naturally-occurring composite materials. That is to say, rocks are not typically chemically or structurally homogeneous materials, but are instead aggregates of different phases having different chemical compositions and structures. For example, rocks typically include multiple different mineral or mineraloid (i.e. non-crystalline mineral-like substances, such as opal or obsidian) phases, and may also contain organic matter, as well liquids (such as water or hydrocarbons) trapped in pores.

Silica (i.e. SiO₂) is a common component of rocks, particularly sedimentary rocks, and may be present in several different forms (e.g. as quartz, amorphous silica or cristobalite). Sedimentary rocks are rocks which were formed at or near the Earth's surface by the accumulation and lithification of material. The material from which sedimentary rocks formed may have been transported into a sedimentary basin from its surroundings by rivers or wind (i.e. allogenic material) or may have been generated where it is now found (i.e. authigenic material).

Accordingly, the silica present in sedimentary rocks can be classified in terms of its origin. Terrigenous or detrital silica in a sedimentary rock is silica incorporated in detrital sedimentary grains in the rock. Detrital sedimentary grains derive originally from weathered material transported into the basin from its hinterland by wind or water (i.e. it is allogenic in origin). Excess silica in a sedimentary rock is silica present in the rock not incorporated in detrital sedimentary grains. Excess silica in sedimentary rocks can be further categorised as biogenic-excess silica or authigenic-excess silica. Biogenic-excess silica was formed by (re)precipitation of silica liberated from prehistoric biota (such as diatoms, radiolaria, silicoflagellates and siliceous sponges) by chemical weathering. Authigenic-excess silica was formed by (re)precipitation of silica liberated from detrital sedimentary material by chemical weathering. Both biogenic-excess silica and authigenic-excess silica form part of the silica (typically quartz) cement or matrix material which surrounds and holds together the detrital grains in the rock.

Hydrocarbon explorers have found that the quality of hydrocarbon reservoirs in a region correlates with the type of silica deposits in that region. For example, relatively higher proportions of excess silica, in comparison to terrigenous/detrital silica, in the rock are associated with increased hydrocarbon production from hydrocarbon wells. Rock parameters such as the porosity and/or mechanical properties (e.g. Young's modulus) of rocks have also been found to depend on the relative amounts of biogenic-excess silica and authigenic-excess silica in the rock. For example, biogenic-excess silica is typically more brittle than authigenic-excess silica and therefore can be easier to fracture, for example when forming hydrocarbon wells (such as lateral wells used to extract hydrocarbons from unconventional sources such as tight rock formations) by hydraulic fracturing. Knowledge of the silica content of rock samples extracted from a region can therefore help in identifying the best locations for drilling well bores and in determining the properties of subterranean rock strata which can be used, for example, in the development and interpretation of seismic models and in the calculation of rock strength, and therefore in the calculation of the pressure environments required to fracture rock (e.g. by hydraulic fracturing) or to maintain rock fractures (whether man-made or naturally occurring).

The mineralogical composition of rocks extracted from hydrocarbon wells can be determined precisely in the laboratory, for example using methods such as quantitative X-ray diffraction (QXRD). The presence of organic phases can also be determined using combustion or pyrolysis analysis methods (e.g. using a LECO instrument for combustion analysis or a Rock-Eval instrument for pyrolysis analysis). The origins of silica in rocks can also be studied by analysing thin sections or using silicon isotope geochemical methods.

However, such analytical methods typically require the use of core samples, which can be difficult and expensive to obtain. For example, it may not be possible to extract core samples from a sufficient number of different sampling locations along a hydrocarbon well path to obtain an accurate picture of the variation in rock composition along the well. The compositional analysis of core samples using standard laboratory methods such as QXRD can also be time-consuming.

One alternative to the use of core samples is to carry out the required compositional analysis measurements on cuttings samples. Cuttings samples are samples of the drill cuttings obtained when a well is drilled; drill cuttings are typically small, broken pieces of rock produced by the drilling action and brought to the surface in the drilling mud. Cuttings samples are therefore typically plentiful, as well as easy and inexpensive to obtain. Cuttings are typically examined as part of mud logging (i.e., well logging) analysis, which includes observation of the cuttings, microscopic examination and basic chemical analysis. It is, however, generally understood that cuttings samples are not suitable for detailed compositional analysis, for example by QXRD, combustion analysis or pyrolysis. Nevertheless, the present inventors have developed methods which can be used to determine silica content of rocks and, in particular, rock cuttings samples.

### Terrigenous Silica and Excess Silica Content

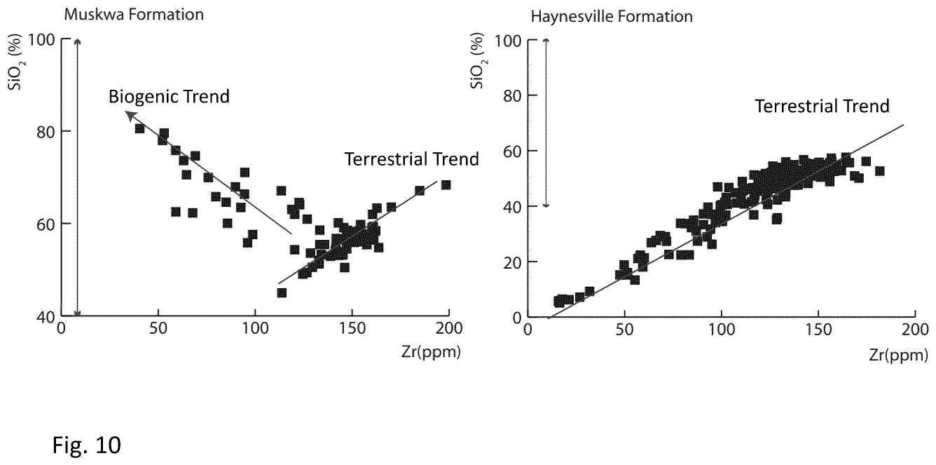

It has been found that the terrigenous/detrital silica content of sedimentary rocks correlates with the abundance of the trace element zirconium (Zr) in the rock. For example, RATCLIFFE et al., Unconventional Methods For Unconventional Plays: Using Elemental Data To Understand Shale Resource Plays, Part 2, PESA News Resources, April/May 2012 (which is hereby incorporated by reference in its entirety) identified a positive linear relationship between the terrigenous (i.e. terrestrial) silica content and the zirconium content of shale rock samples extracted from the Haynesville formation in the United States of America. This trend is visible in FIG. 10, which also shows biogenic and terrestrial trends in silica and zirconium content in samples obtained from the

Muskwa formation in Canada. This trend is understood on the basis that zirconium in sedimentary rocks derives predominantly or entirely from detrital fragments of older rocks (and, in particular, zircon-containing rocks) incorporated in the sedimentary rock and that the abundance of zirconium in sedimentary rocks could not have been enriched significantly by biological activity, nor by (re)precipitation of dissolved silica from ancient bodies of water. Accordingly, it can be assumed that sedimentary rock material containing zirconium is terrigenous in origin.

The present inventors have therefore realised that the abundance of zirconium in sedimentary rocks can be used as a proxy for quantifying the amount of the rock material which is terrigenous/detrital in origin (i.e. which derived from the basin hinterland). Moreover, since, by definition, all of the silica in a sedimentary rock is either terrigenous/detrital or excess in nature (in particular, because excess silica is defined as being that silica which is not terrigenous in origin), the abundance of zirconium in sedimentary rocks can be used to quantify the amount of excess silica present in the rock.

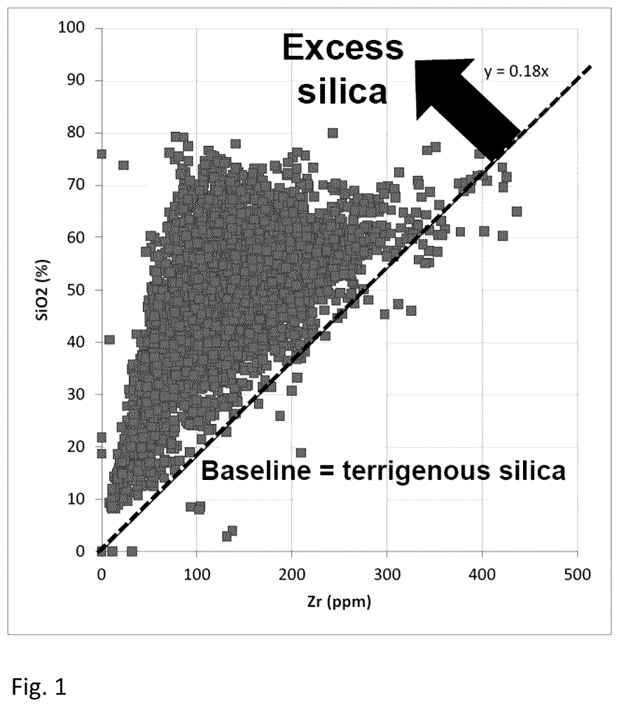

For example, FIG. 1 shows a plot of total silica content (in wt. %) relative to zirconium content (in ppm) for a plurality of different rock cuttings samples obtained from a sedimentary basin. Both the silica content and the zirconium content were measured using X-ray fluorescence (XRF). The data points were found to cluster along or above a straight line defined in FIG. 1 as [SiO₂]ₜₑᵣᵣ=0.18 [Zr], where [SiO₂]ₜₑᵣᵣ is the terrigenous silica content in wt. % and [Zr] is the Zr content in ppm. This straight line defines a relationship between the terrigenous silica content and the zirconium content for sedimentary rocks in the area. Data points lying on the line correspond to samples in which effectively all of the silica present is of terrigenous origin, while data points lying above the line correspond to samples containing both terrigenous silica and excess silica.

The present inventors have realised that the location of a data point in FIG. 1 can be used to quantify the amount of terrigenous silica and the amount of excess silica present in the sample. In particular, the amount of excess silica in the sample is given by the vertical distance (i.e. measured parallel to the [SiO₂] axis) between the sample data point and the straight line, while the amount of terrigenous silica in the sample can be found by subtracting the amount of excess silica from the total amount of silica in the sample (which also corresponds to the [SiO₂] value at the point of intersection between the terrigenous silica boundary line and a vertical line drawn through the sample point parallel to the [SiO₂] axis). Accordingly, it is possible to determine the amounts of terrigenous silica and excess silica graphically.

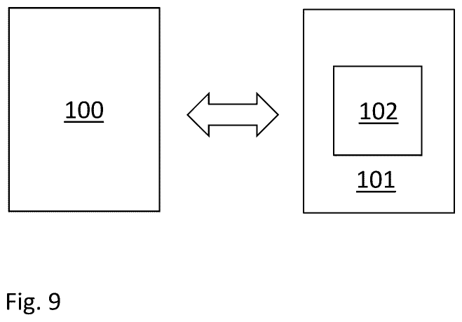

It will be appreciated that the amount of terrigenous silica in a sample can also be calculated directly based on the equation of the straight line, [SiO₂]ₜₑᵣᵣ=0.18 [Zr], and the known Zr content of the sample. The amount of excess silica in the sample can then be calculated as the difference between the known total silica content and the calculated terrigenous silica content. The skilled person will appreciate that such calculations are suited to automation and implementation in computer software (for example, computer software 102 stored on a computer-readable medium 101, for execution by a computer processor 100, as shown in FIG. 9).

In general, the equation of the bounding terrigenous silica line will be different for different sedimentary basins and, potentially, for different areas within a given basin. Accordingly, it is necessary to determine the equation of the terrigenous silica line (i.e. the relationship between amounts of terrigenous silica and amounts of zirconium) based on a plurality of different samples taken from a basin (or a specific area within the basin). That equation can then be used to determine the proportions of terrigenous and excess silica in subsequent samples obtained from the same basin (or specific area within the basin) on the basis of measurements of the zirconium content and the total silica content.

### Biogenic-Excess and Authigenic-Excess Silica Content

As discussed hereinabove, excess silica in sedimentary rocks is made up of biogenic-excess silica and authigenic-excess silica.

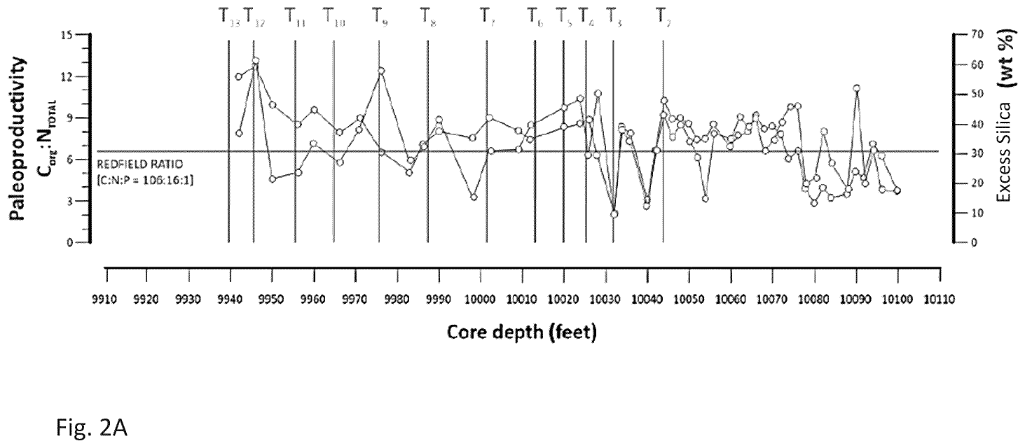

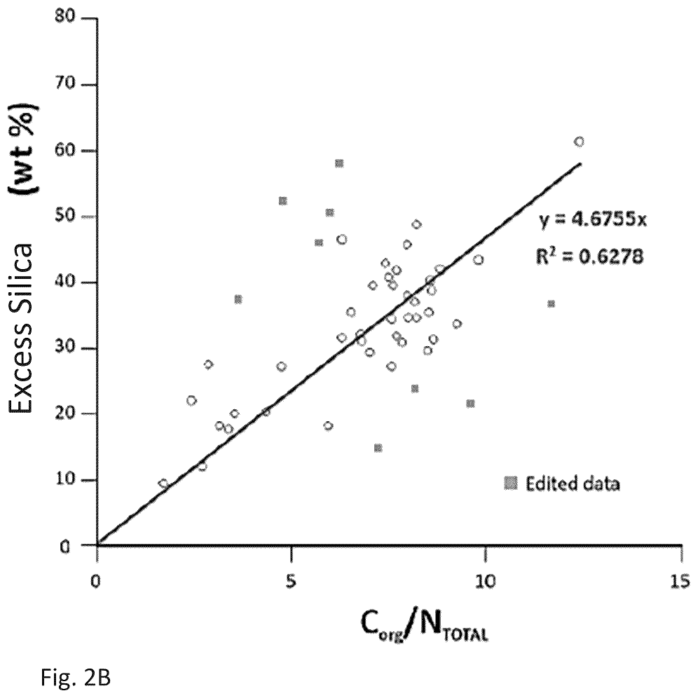

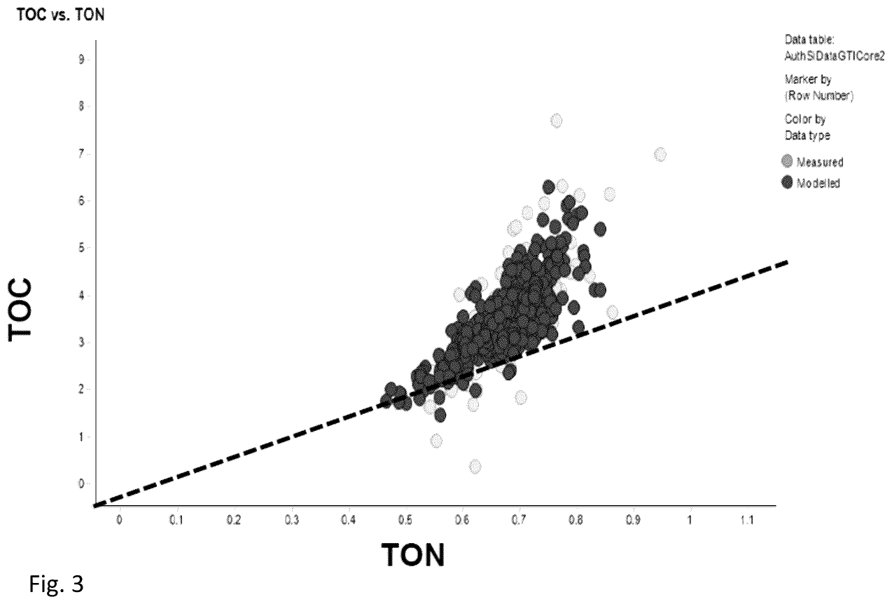

It has been found that the excess silica content of sedimentary rocks, which contain biogenic-excess silica, correlates with the ratio of organic carbon to nitrogen (i.e. the C/N ratio) in the rock. For example, FIG. 2A and FIG. 2B show how the excess silica content of rock samples taken from a well in the Permian Basin correlates with the measured C/N ratio as a function of core depth. This trend is understood on the basis that higher C/N ratios are associated with periods of increased primary paleo-productivity and, therefore, a greater abundance of siliceous organisms (such as Radiolaria) within the paleo-water column. For example, FIG. 3 shows the relationship between total organic carbon (TOC) content and total organic nitrogen (i.e. nitrogen having its origin in living material) content (TON) for a plurality of rock samples extracted from the Laredo formation in Texas (in FIG. 3, “Measured” refers to TOC and TON contents measured directly using e.g. LECO TOC analysis, while “Modelled” refers to TOC and TON contents estimated on the basis of suitably calibrated Fourier-transform infra-red (FTIR) spectroscopic measurements). The dashed baseline indicates that there is no enrichment in organic carbon at low C/N values; there is a base level of C/N that is not related to primary paleo-production in waters. Accordingly, it can be assumed that more of the excess silica in sedimentary rocks having higher C/N ratios is biogenic in origin, while more of the excess silica in sedimentary rocks having lower C/N ratios is authigenic-excess silica.

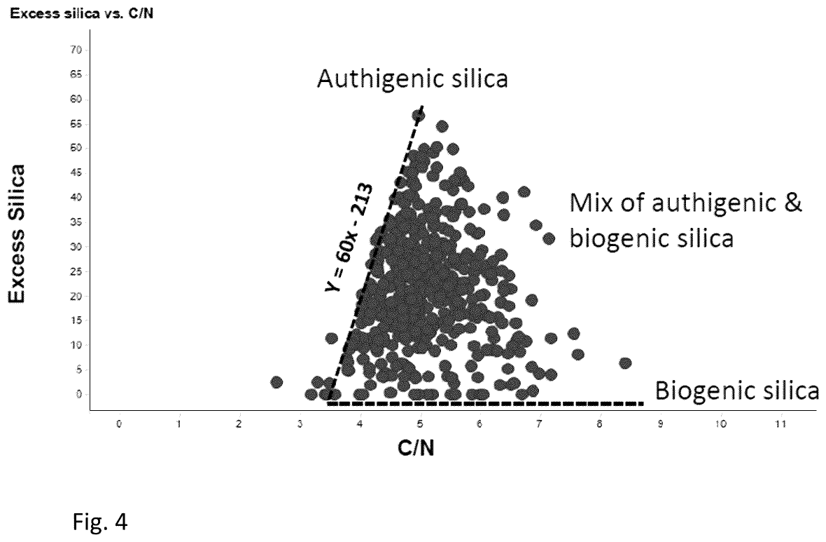

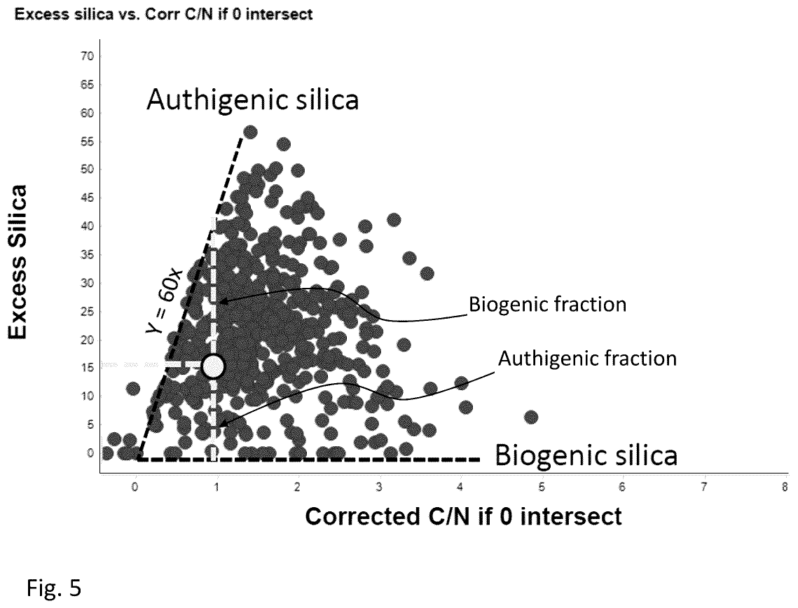

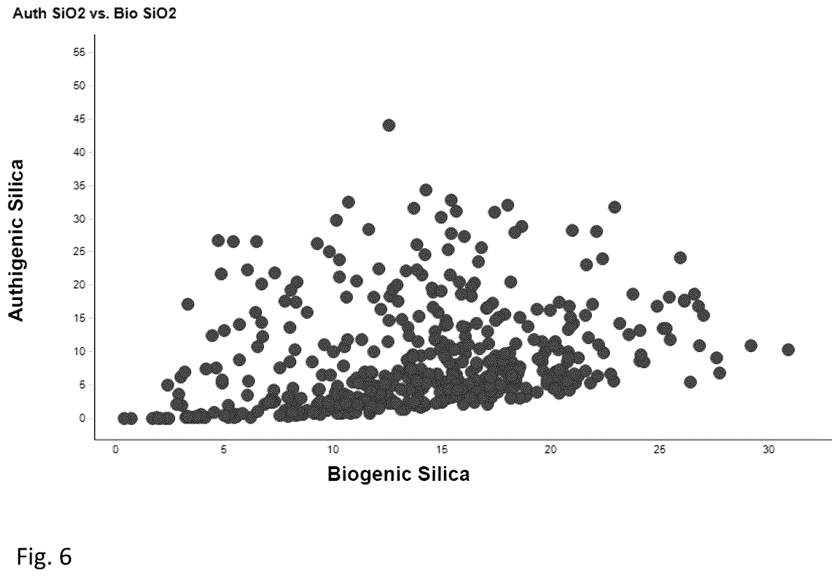

The present inventors have therefore realised that the C/N ratio can be used as a proxy for quantifying the amount of the excess silica in a rock sample which is biogenic in origin and the amount which is authigenic in origin. For example, FIG. 4 shows a plot of excess silica values as a function of the C/N ratio for a plurality of rock samples extracted from a particular region. Biogenic and authigenic boundary lines are defined. The biogenic boundary line lies close to, and almost parallel to, the C/N axis, although in practice this line typically has a small positive slope. The authigenic boundary line has a steeper slope. More biogenic-excess silica rich samples lie closer to the biogenic boundary line than those in closer proximity to the sloping authigenic boundary line. The proportions of the excess silica in a given sample which are biogenic-excess and authigenic-excess can be calculated by measuring the vertical distance (i.e. parallel to the excess silica axis) of the corresponding data point from the two boundary lines, the ratio of biogenic-excess to authigenic-excess silica in the sample being given by the ratio of the distance to the authigenic line to the distance to the biogenic line. The absolute amount of biogenic-excess silica and the absolute amount of authigenic-excess silica in the sample can be calculated based on this ratio and on the known total amount of excess silica in the sample. This calculation can be carried out graphically, as shown in FIG. 5, in which all of the data points have been translated such that the biogenic and authigenic boundary lines intersect at the origin for simplicity. Accordingly, it is possible to determine the amounts of biogenic-excess silica and authigenic-excess silica graphically. As shown in FIG. 6, the sample data can then be plotted as a function of the proportions of authigenic-excess and biogenic-excess silica present.

It will also be appreciated that the amounts of biogenic-excess silica and authigenic-excess silica in a sample can also be calculated directly based on the equations of the biogenic and authigenic boundary lines and the known excess silica content for a sample. The skilled person will appreciate that such calculations are suited to automation and implementation in computer software (for example, computer software 102 stored on a computer-readable medium 101, for execution by a computer processor 100, as shown in FIG. 9).

In general, the equations of the authigenic and biogenic boundary lines will be different for different sedimentary basins and, potentially, for different areas within a given basin. Accordingly, it is necessary to determine the equations of the lines (i.e. the relationships between amounts of biogenic-excess and authigenic-excess silica and the C/N ratio) based on a plurality of different samples taken from a basin (or a specific area within the basin). Those equations can then be used to determine the proportions of biogenic-excess and authigenic-excess silica in subsequent samples obtained from the same basin (or specific area within the basin) on the basis of measurements of the excess silica content and the C/N ratio.

### Compositional Analysis

The methods discussed hereinabove for determining the proportions of terrigenous silica and excess silica, and in turn biogenic-excess silica and authigenic-excess silica, in rock samples require as input measurements the amount of silica (i.e. the total amount of silica of all types), the amount of zirconium, the amount of organic carbon and the amount of nitrogen in the rock samples. These quantities may be determined by precise laboratory methods such as X-ray diffraction (XRD) (e.g. quantitative X-ray diffraction (QXRD)), combustion analysis (e.g. using a LECO analyser available from the LECO Corporation, Saint Joseph, Michigan, USA), pyrolysis analysis (e.g. Rock-Eval pyrolysis using a Rock-Eval analyser available from Vinci Technologies SA, Nanterre, France), electron probe microanalysis, etc. Such methods typically require the use of core samples.

However, the inventors have also found that spectroscopic methods such X-ray fluorescence (XRF) may be used to determine the silica content and zirconium content of rock samples including cuttings samples. XRF is a well-known spectroscopic technique for obtaining an X-ray spectrum from a sample, the X-ray spectrum being reflective of the elemental or chemical composition of the sample. In XRF, a sample is exposed to X-rays or gamma rays of sufficient energy to cause ejection of one or more inner orbital electrons from atoms in the sample. Electrons from higher orbitals then fall into the empty lower orbitals and release energy in the form of photons of characteristic wavelengths which are typically in the X-ray range. The X-ray spectrum generated by the sample is analysed in order to identify the species present, and the intensity of the radiation emitted at each characteristic wavelength can be used to determine the amount of each species in the sample. The silica content of a sample may be obtained, for example, by first measuring the silicon content of the sample by XRF and then calculating the silica content of the sample, for example assuming that all silicon is present in the form of silica. Portable, hand-held XRF analysers are available. Suitable example XRF analysers include the Niton XL5 Handheld XRF Analyzer (available from Thermo Fisher Scientific Inc, USA), which is a hand-held device, and the SPECTRO XEPOS or SPECTROSCOUT (available from SPECTRO Analytical Instruments GmbH, Germany), which are benchtop devices.

The inventors have also developed a spectroscopic method for determining the composition of rock samples such as cuttings samples. While the skilled person will appreciate that the method may be implemented using many different kinds of spectroscopy, the discussion which follows is focused on the particular example of Fourier-transform infra-red spectroscopy (FTIR).

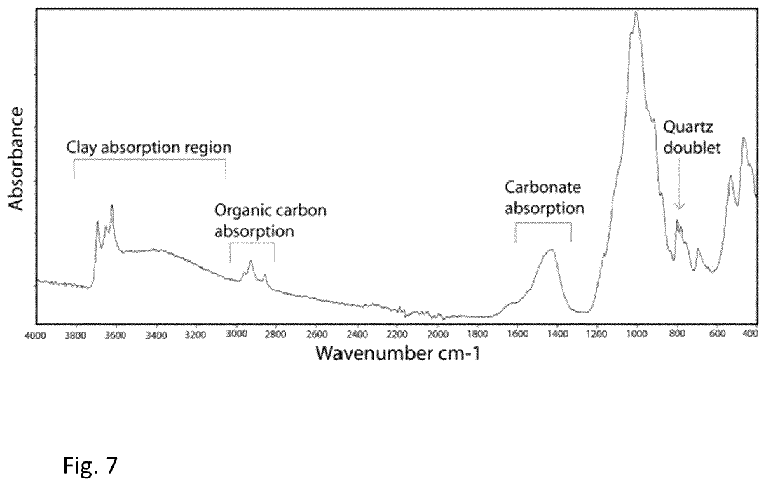

FTIR is a well-known spectroscopic technique for obtaining an infra-red absorption spectrum from a sample, the IR spectrum being reflective of the abundance of particular molecular bonds within the sample. FTIR typically makes use of attenuated total reflection (ATR). The sample (which may be powdered) is held in contact with an optically dense crystal having a high refractive index, and an infra-red beam is directed through the crystal at an angle sufficient to cause total internal reflection within the crystal, thereby generating an evanescent wave which extends beyond the surface of the crystal and into the sample. In regions of the infra-red spectrum where the sample absorbs energy, the evanescent wave will be attenuated or otherwise altered. Attenuated signal from each evanescent wave is passed back into the main infra-red beam which exits the crystal and is detected in the IR spectrometer, thereby generating an infra-red spectrum. For example, FIG. 7 shows an example FTIR spectrum obtained from a rock sample, illustrating the regions of the spectrum associated with infra-red attenuation due to the presence of common mineralogical phases such as clays, quartz and carbonates, as well as a region associated with organic carbon.

Although it is possible to identify the presence of organic phases in FTIR spectra (as seen in FIG. 7), spectroscopic methods are again not conventionally considered suitable for determining the amount of organic phases present in a quantitative manner. Instead, quantitative methods such as combustion analysis (e.g. TOC combustion analysis (for example, using a LECO carbon analyser available from the LECO Corporation, Saint Joseph, Michigan, USA)) or pyrolysis analysis (e.g. Rock-Eval pyrolysis using a Rock-Eval analyser available from Vinci Technologies SA, Nanterre, France) methods are traditionally used to determine the organic phase composition of rock samples. For example, a combustion analyser may be used to combust the organic carbon remaining in a rock sample following chemical removal of inorganic carbon (i.e. carbonates), and to measure the amount of carbon dioxide produced, thereby providing a measurement to the total amount of organic carbon in the sample. Combustion analysis may also be used to determine the nitrogen content of rock samples. Again, however, such methods typically require the use of core samples and are also destructive.

However, the present inventors have found that quantitative measurements of carbon content and nitrogen content can be achieved by using a spectroscopic model fitted to a reference dataset compiled using, for example, combustion (e.g. LECO) analysis methods as applied to reference rock samples from the region.

In the new calibrated spectroscopic method developed by the inventors, a training data set of carbon and/or nitrogen content data is compiled using, e.g., combustion (e.g. LECO) analysis methods. The reference training data set is compiled by measuring the carbon and/or nitrogen content data for a plurality of different core samples of rock taken from a region. It has been found that a minimum of around 30 samples is typically required to build a representative training data set.

The reference training data set includes, for example, measurements of the total organic carbon content and/or the nitrogen content in each sample. In addition, an FTIR spectrum (or other suitable spectroscopic spectrum) is obtained for each sample using an FTIR instrument (for example, an ALPHA FTIR spectrometer available from Bruker Corporation, Billerica, MA, United States of America) to compile an FTIR training data set. The FTIR training data set is then fit to the reference training data set using a multivariate statistical approach such as a least squares regression methodology (for example, using the OPUS spectroscopy software available from Bruker Corporation, Billerica, MA, United States of America. The FTIR spectra are matched to the carbon and nitrogen components in the reference training data set and used to build a calibration model (also referred to as a chemometric model). In particular, multivariate calibration algorithms such as Partial Least Squares (PLS), as implemented in the OPUS spectroscopy software, can be used to correlate spectral intensity (e.g. absorbance values) in specified FTIR wavelength regions (i.e. peak areas in an FTIR spectrum) with concentration values for constituents in the reference training data set.

In the method developed by the inventors, cross-validation techniques are used to determine the quality of the calibration. Cross validation is a statistical process whereby a model is validated using the data points within it. For example, in a model fit to data obtained from 30 samples, cross validation could involve using the model to predict the results which would be expected for each of the 30 samples, one at a time, based on the data obtained from the other 29 samples in the set.

Once the calibration model has been built, it can be used to determine the carbon and/or nitrogen content of an unknown rock sample based on a measured FTIR spectrum. This method is particularly suitable for the compositional analysis of large volumes of cuttings samples extracted from hydrocarbon wells, in particular due to the speed of the FTIR analysis and the subsequent comparison with the calibration model. For example, the inventors have found that the composition of a cuttings sample can be analysed within about 30 seconds, and an atmospheric calibration of only about 30 seconds is required between sequential sample analyses. Accordingly, the method is suited to the compositional analysis of cuttings samples from hydrocarbon wells during drilling.

In a variant of this method, an alert is triggered if a measured FTIR spectrum cannot be matched sufficiently accurately to the calibration model. Accuracy of a match can be assessed in terms of the distance of a measured FTIR spectrum from the calibration model, for example in terms of the Mahalanobis distance (which is a multidimensional generalisation of the method of using the number of standard deviations from the mean to quantify distance). If an alert is triggered, new reference carbon and/or nitrogen content measurements are performed on the and the resultant data, as well as the measured FTIR spectrum, are incorporated into the existing calibration model, thereby extending the range of calibration. This enables the model to be developed and improved on-the-fly.

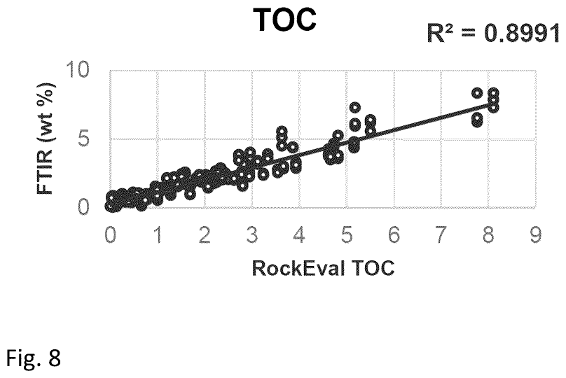

FIG. 8 illustrates how accurately the FTIR measurements can be fit to Rock-Eval pyrolysis reference measurements of the total organic carbon (TOC) content when training the calibration model. As can be seen, there is a strong correlation (as quantified by the coefficient of determination, R²) between the FTIR results and the Rock-Eval measurements.

It will be understood that the invention is not limited to the embodiments described above and various modifications and improvements can be made without departing from the concepts described herein. Except where mutually exclusive, any of the features may be employed separately or in combination with any other features and the disclosure extends to and includes all combinations and sub-combinations of one or more features described herein.

1. A method comprising:
providing spectroscopic measurement data, such as infra-red spectroscopic measurement data, obtained from a plurality of reference rock samples;
providing corresponding carbon and/or nitrogen compositional measurement data obtained from the same plurality of reference rock samples, wherein carbon and/or nitrogen compositional measurement data obtained from a reference rock sample is indicative of an amount of carbon and/or an amount of nitrogen in the reference rock sample; and
fitting a spectroscopic calibration model to the spectroscopic measurement data and the corresponding carbon and/or nitrogen compositional measurement data, wherein the spectroscopic calibration model defines a relationship between the spectroscopic measurement data, such as the infra-red spectroscopic measurement data, and the corresponding carbon and/or nitrogen compositional measurement data for the plurality of reference rock samples.

2. The method according to claim 1, wherein the carbon and/or nitrogen compositional measurement data obtained from a reference rock sample comprises a measurement indicative of an amount of carbon and/or a measurement indicative of an amount of nitrogen in the reference rock sample.

3. The method according to claim 2, wherein the measurement indicative of the amount of carbon and/or the measurement indicative of the amount of nitrogen in the reference rock sample are combustion analysis measurements.

4. The method according to claim 1, wherein the method is carried out by a computer.

5. A computer program comprising instructions which, when the program is executed by a computer, cause the computer to carry out the steps of the method of claim 1.
